(19) United States
(12) Patent Application Publication
(10) Pub. No.: US 2025/0040848 A1
(43) Pub. Date: Feb. 6, 2025

(54) Urinary Output Monitoring System

(71) Applicant: C. R. Bard, Inc., Franklin Lakes, NJ (US)

(72) Inventors: Kuilin Lai, Watkinsville, GA (US); Gregory Mann, Mcdonough, GA (US); Skylar Vander Laan, Decatur, GA (US)

(21) Appl. No.: 18/706,737

(22) PCT Filed: Nov. 9, 2022
(86) PCT No.: PCT/US22/49418

§ 371 Date: May 1, 2024

Related U.S. Application Data
(60) Provisional application No. 63/278,069, filed on Nov. 10, 2021.

(51) Int. Cl. A61B 5/20; A61F 5/44; A61F 5/441; A61F 5/455; A61M 1/00

(52) U.S. Cl. CPC A61B 5/208; A61F 5/4405; A61F 5/441; A61F 5/455; A61M 1/80

(57) ABSTRACT

A urinary output monitoring system including a urine collection assembly coupled with an automated urinary output monitoring device. The urine collection assembly includes a drainage tube, an accumulator coupled with the drainage tube, and a urine collection container fluidly coupled with the accumulator. The automated urinary output monitoring device operatively is coupled with the urine collection container. Logic determines a volume of urine collected in the container based on a signal from a load cell of the monitoring device. A vacuum pump coupled with the accumulator generates a flow of air that the drags the urine along the drainage tube to the accumulator. The accumulator separates the flow of air from the urine. The flow of air enters the drainage tube by way of a female external urinary catheter.

### PRIORITY

This application claims the benefit of priority to U.S. Provisional Application No. 63/278,069, filed Nov. 10, 2021, which is incorporated by reference in its entirety into this application.

### BACKGROUND

Current urinary output monitoring systems capture voided urine from patients over time. These systems may include a urinary catheter in urine communication with a urine collection container configured to capture urine output by gravity flow. However, dependent loops can form within the tubing, leading to an increase in catheter assisted urinary tract infections in patients. Furthermore, dependent loops can lead to inaccurate urine output measurements. It would be beneficial to the patient to have a urinary output monitoring system that removes dependent loops from the system and is adaptable to current urinary output monitoring systems. Such a system would lead to accurate urine output measurements and may reduce the likelihood of a patient experiencing a catheter assisted urinary tract infection. Disclosed herein is a urinary output monitoring system and method that address the foregoing.

### SUMMARY

Disclosed herein is a urinary output monitoring system, that, according to some embodiments, includes a urine collection assembly coupled with an automated urinary output monitoring device. The urine collection assembly includes a drainage tube defining a distal end and a proximal end, an accumulator coupled with the drainage tube at the proximal end, and a urine collection container fluidly coupled with the accumulator. The automated urinary output monitoring device is operatively coupled with the urine collection container, and the automated urinary output monitoring device includes a console in communication with a sensor of the system. The console includes a processor and memory having logic stored thereon that, when executed by the processor, performs operations of the system including determining a volume of urine collected within the urine collection container. The system further includes a vacuum pump fluidly coupled with the accumulator, such that air is removed from the accumulator by the vacuum pump. The air removed from the accumulator causes a flow of air combined with a flow of urine along the drainage tube from the distal end to the accumulator, and the accumulator is configured to separate the flow of air from the flow of urine.

In some embodiments, the urine collection assembly further includes a urinary catheter coupled with the drainage tube at the distal end. In some embodiments, the flow of air passes through the urinary catheter and in some embodiments, the urinary catheter is an external female urinary catheter.

In some embodiments, the accumulator defines a cavity including an upper cavity portion and a lower cavity portion. In such embodiments, the flow of air passes into and out of the upper cavity portion and only the flow of urine passes into and out of the lower cavity portion.

In some embodiments, the accumulator is attached to the automated urinary output monitoring device such that the accumulator is disposed in a vertical orientation.

In some embodiments, the sensor includes a load cell of the automated urinary output monitoring device. The load cell is operatively coupled with the urine collection container and the load cell is configured to determine a gravitational load defined by urine collected within the urine collection container. In such embodiments, determining a volume of urine collected within the urine collection container includes determining the volume of urine based on the gravitational load.

In some embodiments, the urine collection assembly further includes a one-way valve disposed in line with a lumen extending between the accumulator and the urine collection container, where the one-way valve is configured to prevent fluid flow from the urine collection container toward the accumulator.

In some embodiments, the urine collection container is a rigid container, and in some embodiments, the accumulator is rigidly coupled with the urine collection container. In some embodiments, the urine collection container includes an air vent configured to a define an atmospheric pressure within the urine collection container.

In some embodiments, the sensor includes a flow meter disposed in line with the lumen extending between the accumulator and the urine collection container, where the flow meter is configured to measure a flow rate of urine passing from the accumulator to urine collection container, and where determining a volume of urine collected within the urine collection container includes determining the volume of urine based on the flow rate. In some embodiments, the urine collection container includes a detachable lid.

Also disclosed herein is urinary output monitoring system method that, according to some embodiments, includes receiving urine from a patient within a drainage tube coupled with and extending proximally away from a urinary catheter. The method further includes (i) establishing a flow of air proximally along the drainage tube between the urinary catheter and an accumulator disposed at a proximal end of the drainage tube and (ii) combining the urine with the flow of air such that the flow of air causes the urine to flow proximally along the drainage tube toward the accumulator. The method further includes separating the flow of air from the urine within the accumulator and collecting the urine within a urine collection container located downstream of the accumulator.

In some embodiments of the method, establishing the flow of air includes activating a vacuum pump fluidly coupled with the accumulator.

In some embodiments, the method further includes (i) measuring a gravitational load defined by urine collected within the urine collection container and (ii) determining a volume of the urine collected within the urine collection container based on the gravitational load.

In some embodiments of the method, the flow of air enters the drainage tube by way of the urinary catheter. In some embodiments of the method, the urinary catheter is an external female urinary catheter, and in some embodiments of the method, the flow of air enters the urinary catheter via a non-sealing interface between the urinary catheter and the patient.

Also disclosed herein is a urine collection assembly that, according to some embodiments, includes an accumulator defining a closed cavity including an upper cavity portion and a lower cavity portion, and the accumulator includes an air port in direct fluid communication with the upper cavity portion. The assembly further includes (i) a first drainage tube defining a first distal end and a first proximal end, where the first drainage tube is coupled with the accumulator at the first proximal end so as to be in direct fluid communication with the upper cavity portion; (ii) a second drainage tube defining a second distal end and a second proximal end, where the second drainage tube is coupled with the accumulator at the second distal end so as to be in direct fluid communication with the lower cavity portion; and (iii) a urine collection container coupled with the second drainage tube at the second proximal end. The air port is configured to couple with an air hose of a vacuum pump such that air flow defined by the vacuum pump flows (i) proximally along the first drainage tube and (ii) into and out of the upper cavity portion.

In some embodiments of the assembly, the urine collection container is configured to operatively couple with a load cell of an automated urinary output monitoring device such that urine collected within the urine collection container defines a gravitational load applied to the load cell. The automated urinary output monitoring device includes a console in communication with the load cell, and the console includes a processor and memory having logic stored thereon. The logic, when executed by the processor, performs operations of the automated urinary output monitoring device that include determining a volume of the urine collected within the urine collection container based on the gravitational load.

In some embodiments, the assembly further includes a female external catheter coupled with the first drainage tube at the first distal end such that, during use, urine excreted by a patient (i) enters the female external catheter, (ii) flows proximally along the first drainage tube, and (iii) enters the accumulator.

In some embodiments, during use, the air flow defined by the vacuum pump causes the urine to the flow proximally along the first drainage tube.

In some embodiments, during use, the urine excreted by a patient (i) separates from the flow of air within the accumulator, (ii) falls from the upper cavity portion to the lower cavity portion, (ii1) flows proximally along the second drainage tube, and (iv) enters the urine collection container.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A illustrates a urinary output monitoring system, in accordance with some embodiments;

FIG. 1B illustrates a cross-sectional view of the accumulator of the urine collection assembly of FIG. 1A, in accordance with some embodiments;

FIG. 2 illustrates a second embodiment of an urinary output monitoring system, in accordance with some embodiments;

FIG. 3 is an illustration of the system of FIG. 1A in use, in accordance with some embodiments; and

FIG. 4 illustrates a flow chart of the exemplary method of the monitoring urinary output system of FIG. 1A, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

The terms “proximal” and “distal” refer to opposite ends of a medical device, including the devices disclosed herein. As used herein, the proximal end of a medical device or component is the end nearest a practitioner during use, while the distal end is the opposite end. For example, the proximal end of a drainage tube is defined as the end closest to the practitioner during utilization of the drainage tube. The distal end is the end opposite the proximal end, along the longitudinal direction of the drainage tube.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Unless defined otherwise, all technical and scientific terms used herein have the same meaning as commonly understood by those of ordinary skill in the art. As used herein, the term “fluid” means a liquid or a gas, such as air or urine, for example.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

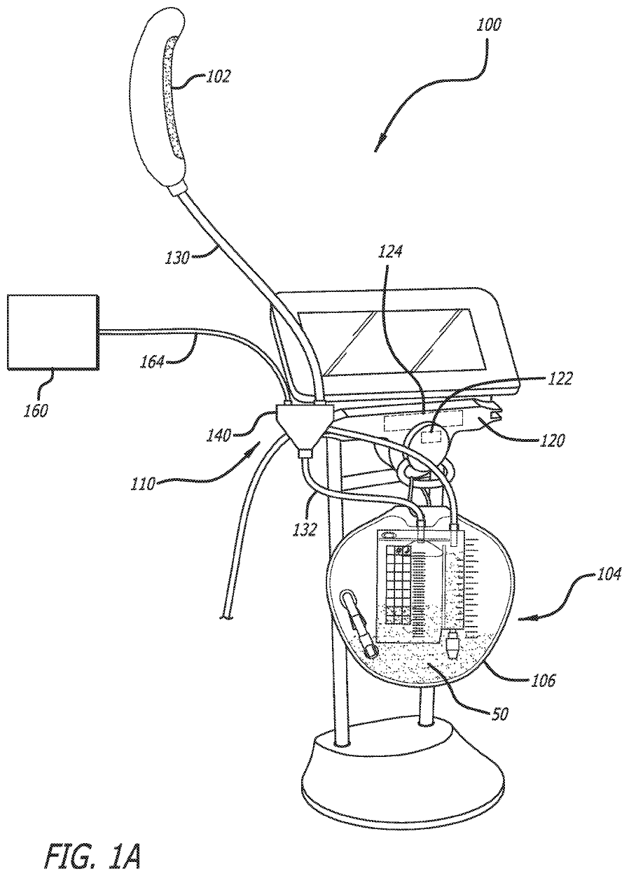

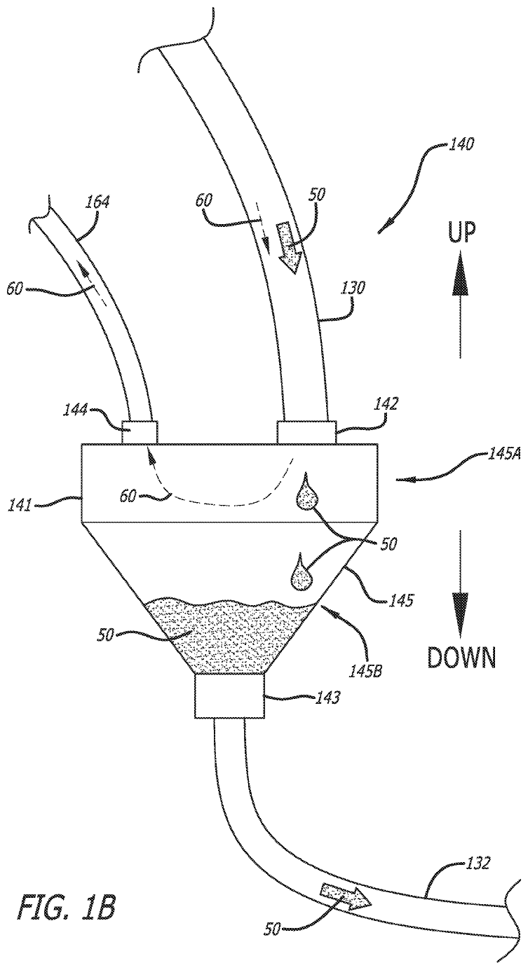

FIG. 1A is an illustration of a urinary output monitoring system 100, in accordance with some embodiments. The urinary output monitoring system (“system”) 100 generally includes a urine collection assembly 110 operatively coupled with an automated urinary output monitoring device 120. The system 100 is generally configured to receive and collect urine output from a patient and generate metrics related to the urine output. The system 100 is further configured to actively displace urine proximally along a drainage tube, such as urine that may accumulate within a dependent loop of the drainage tube, for example.

The urine collection assembly 110 includes fluid components that when assembled together are configured to receive urine 50 from the patient and direct the urine to a urine collection container 104, such as the urine collection bag 106. In some embodiments, urine collection assembly 110 may include the urinary catheter 102, where the urinary catheter 102 may be an external female urinary catheter. The urine collection bag 106 is detachably coupled to the automated urinary output monitoring device 120. In some embodiments, the urine collection bag 106 may be configured to hang from the automated urinary output monitoring device 120.

The automated urinary output monitoring device 120 includes a load cell 122 in communication with a console 124. The load cell 122 is configured measure a load related to urine 50 collected within the urine collection bag 106 when the urine collection bag 106 is coupled to the automated urinary output monitoring device 120. The load cell 122 may be configured to make multiple measurements as the urine collection bag 106 receives the urine 50. In some embodiments, the load measurements may be used to generate a number (e.g., 1, 2, 3 or more) of metrics related to the collection of the urine 50 within the urine collection bag 106, such as a flow rate or a total volume, for example as will be described in more detail herein. In some embodiments, the automated urinary output monitoring device 120 may be free standing, suspended from a stand, or may be coupled to a securing surface, such as a wall, for example. In some embodiments, the console 124 may include a wireless module (not shown) so that the console may be in communication with an external computing device (not shown) such as personal computer, a tablet, or a cell phone, for example. The external computing device may be connected to an electronic medical records system.

The urine collection assembly 110 includes an accumulator 140 placed in line between the urinary catheter 102 and the urine collection bag 106. A first drainage tube 130 extends between the urinary catheter 102 and the accumulator 140, and a second drainage tube 132 extends between the accumulator 140 and the urine collection bag 106. An air hose 164 fluidly couples the accumulator 140 with a vacuum pump 160, where the vacuum pump 160 is generally configured to draw air 60 from the accumulator 140.

FIG. 1B is a detailed illustration of the accumulator 140. The accumulator 140 includes a body 141 defining a closed cavity 145. During use accumulator 140 is disposed in a vertical orientation as shown in FIG. 1B. The cavity 145 defines an upper cavity portion 145A and a lower cavity portion 145B. The first drainage tube 130 is coupled with the accumulator 140 via an inlet port 142 where inlet port 142 establishes fluid communication between the first drainage tube 130 and the upper cavity portion 145A. Similarly, the air hose 164 is coupled with the accumulator 140 via an air port 144 where the air port 144 establishes fluid communication between the air hose 164 and the upper cavity portion 145A. The second drainage tube 132 is coupled with the accumulator 140 via an outlet port 143 where outlet port 143 establishes fluid communication between the second drainage tube 132 and the lower cavity portion 145A. In some embodiments, the accumulator 140 may be constructed of plastic, one or more polymers, or the like. In some embodiments, the accumulator 140 may be extruded, injection molded, 3D printed, or the like. The accumulator 140 may be configured for single use or use with a single patient. In alternative embodiments, the accumulator 140 may be configured for re-use across multiple patients.

During use, a combination of air 60 and urine 50 flow along the first drainage tube 130 away from the patient and enter the accumulator 140 via the inlet port 142. Within the accumulator 140, the air 60 separates from the urine 50. More specifically, the air 60 (i) enters the upper cavity portion 145A via the inlet port 142 and (ii) exits the upper cavity portion 145A via the air port 144. The urine 50 (i) enters the upper cavity portion 145A via the inlet port 142, (ii) separates from the air 60, (iii) falls from the upper cavity portion 145A to the lower cavity portion 145B, and (iv) exits the lower cavity portion 145B via the outlet port 143. In some embodiments, the cavity 145 may include a conical or funnel shape to direct urine 50 with the cavity 145 toward the outlet port 143. The urine 50 flows along the second drainage tube 132 to the urine collection bag 106 due to gravity.

The vacuum pump 160 generally defines an air flow (i.e., a flow of the air 60) along the first drainage tube 130, into and out of the accumulator 140, and along the air hose 164. In some embodiments, the urinary catheter 102 is not sealably coupled with the patient. As such, air 60 may enter the urinary catheter 102 at the catheter-patient interface. As such, both urine 50 and air 60 may enter the first drainage tube 130 and flow along the first drainage tube 130 toward the accumulator 140. The air flow as caused by the vacuum pump 160 may drag the urine 50 along the first drainage tube 130 toward the accumulator 140. In some instances, the urine 50 may flow along the first drainage tube 130 toward the accumulator 140 without the aid of the air flow caused by the vacuum pump 160. For example, the urine 50 may flow along the first drainage tube 130 while the vacuum pump 160 is deactivated.

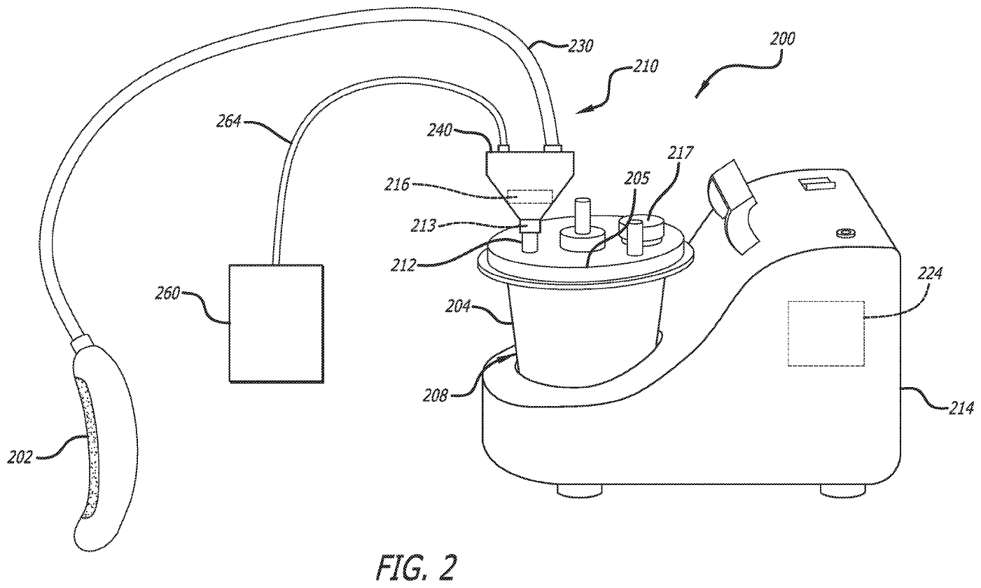

FIG. 2 illustrates another embodiment of a urinary output monitoring system that can, in certain respects, resemble components of the urinary output monitoring system 100 described in connection with FIGS. 1A-1B. It will be appreciated that the illustrated embodiments may have analogous features. Accordingly, like features are designated with like reference numerals, with the leading digits incremented to “2.” For instance, the accumulator is designated as “140” in FIGS. 1A-1B, and an accumulator is designated as “240” in FIG. 2. Relevant disclosure set forth above regarding similarly identified features thus may not be repeated hereafter. Moreover, specific features of the urinary output monitoring system 100 and related components shown in FIGS. 1A-1B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the urinary output monitoring system of FIG. 2. Any suitable combination of the features, and variations of the same, described with respect to the urinary output monitoring system 100 and components illustrated in FIGS. 1A-1B can be employed with the urinary output monitoring system 200 and components of FIG. 2, and vice versa.

The urinary output monitoring system 200 includes the urinary catheter 202 in urine communication with the urine collection container 204 configured to receive urine therein. The urine collection container 204 includes a rigid container 208 configured to receive the urine therein, where the rigid container 208 includes a detachable lid 205. As such, the detachable lid 205 may be separated from the rigid container 208 to enable disposal of the collected urine. In some embodiments, the detachable lid 205 may be detachably coupled to the rigid container 208 through a press fit, a snap fit, a screw fit, an interference fit, or the like. In some embodiments, the detachable lid 205 may include an intake port 212 configured to receive the urine therethrough. In some embodiments, the rigid container 208 may be placed on a docking station 214 configured to stabilize the rigid container 208 during use. The accumulator 240 is placed in line between the urinary catheter 202 and the rigid container 208. In some embodiments, the first drainage tube 230 may provide urine communication between the urinary catheter 202 and the accumulator 240. In some embodiments, the accumulator 140 may be directly attached (e.g., rigidly attached) to the intake port 212. The accumulator 140 is coupled with the vacuum pump 260 via the air hose 264. In some embodiments, the vacuum pump 260 may be a standalone structure, or may be coupled to or integrated into the docking station 214. The accumulator 240 may be placed in the vertical orientation by directly coupling the accumulator 240 to the intake port 212.

In some embodiments, the accumulator 240 may include a flow meter 216 in communication with the console 224, the flow meter 216 is configured to measure the flow or flow rate of urine passing from the accumulator 240 into the rigid container 208. In some embodiments, the console 224 may be integrated into the flow meter 216, may be coupled to the detachable lid 205, or the docking station 214. In some embodiments, the console 224 may be in communication with the external computing device. In some embodiments, the measured flow rate may be used to detect, calculate, or determine the metrics related to the collected urine. In some embodiments, the flow meter 216 may be integrated into the accumulator 240 or may be a separate structure coupled to the accumulator 240. In some embodiments, the intake port 212 may include a one way valve 213 disposed in line with the intake port 212, where the one way valve 213 is configured to prevent fluid (i.e., urine and/or air) from exiting the rigid container 208 via the intake port 212. In some embodiments, the rigid container 208 or the detachable lid 205 may include a vent 217 configured to define an atmospheric pressure within the rigid container 208.

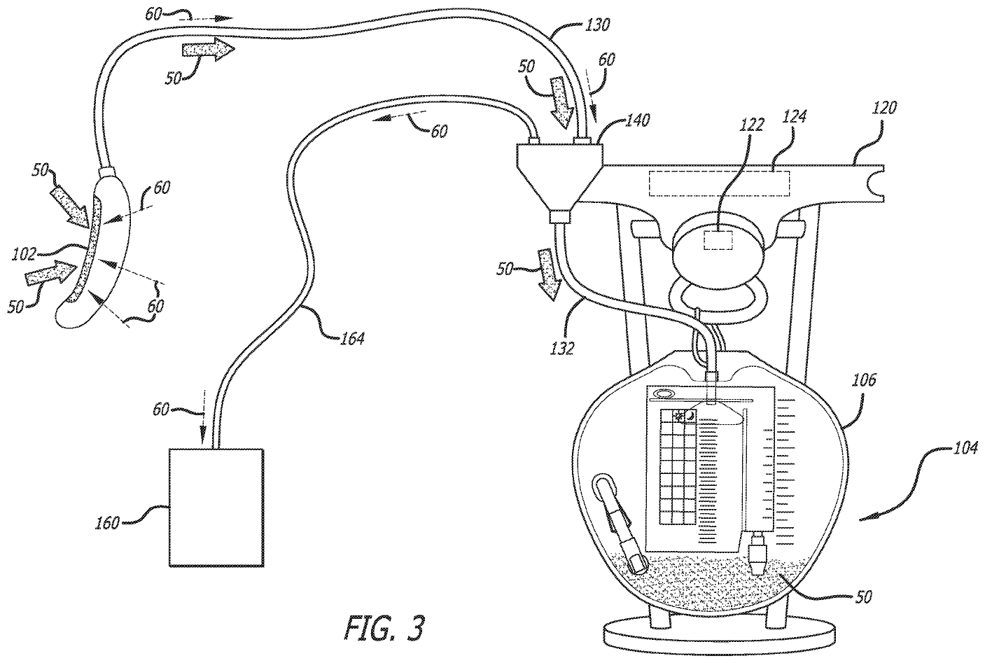

FIG. 3 illustrates the urinary output monitoring system 100 in use. The urine collection assembly 110 is operatively coupled with the automated urinary output monitoring device 120, i.e., the urine collection bag 106 is coupled with the load cell 122. The accumulator 140 is attached to the urinary output monitoring device 120 so that the accumulator 140 is vertically oriented. The vacuum pump 160 is fluidly coupled with the accumulator 140 via the air hose 164.

The urinary catheter 102 is coupled with the patient (not shown) so that urine 50 excreted by the patient enters the urinary catheter 102. The urinary catheter 102 interfaces in a non-sealing fashion so that air 60 enters the urinary catheter 102 at the catheter-patient interface. The vacuum pump 160 is activated to establish the flow of air 60 along the first drainage tube 130 from the urinary catheter 102 to the accumulator 140, thereby dragging the urine 50 along the first drainage tube 130.

The air 60 separates from the urine 50 within the accumulator 140 within the upper cavity portion 145A (see FIG. 1B). The air 60 exits the accumulator 140 (i.e., the upper cavity portion 145A) via the air port 144. The urine 50 falls from the upper cavity portion 145A to the lower cavity portion 145B and exits the accumulator 140 (i.e., the lower cavity portion 145B) via the outlet port 143. The urine 50 flows along the second drainage tube 132 toward the urine collection bag 106 due to gravity.

The urine collection bag 106 collects the urine 50. The urine 50 collected within the urine collection bag 106 defines a gravitational load (e.g., a weight) on the load cell 122. The load cell 122 measures the load and provides an electrical signal to the console 124 in accordance based on the gravitational load defined by the urine 50 collected within the urine collection bag 106.

The logic of the console 124 processes the electrical signal to define a volume of the urine 50 within the collection bag 106. In some embodiments, the logic obtains multiple electrical signals from the load cell 122 to define a number of the metrics related to the collection of the urine 50, such as an instant volume of urine, a total volume of urine, a rate of urine output over a defined time period, a number of urine output excretions, or a time period between urine output excretions, for example. The logic may transmit the metrics to the external computing device.

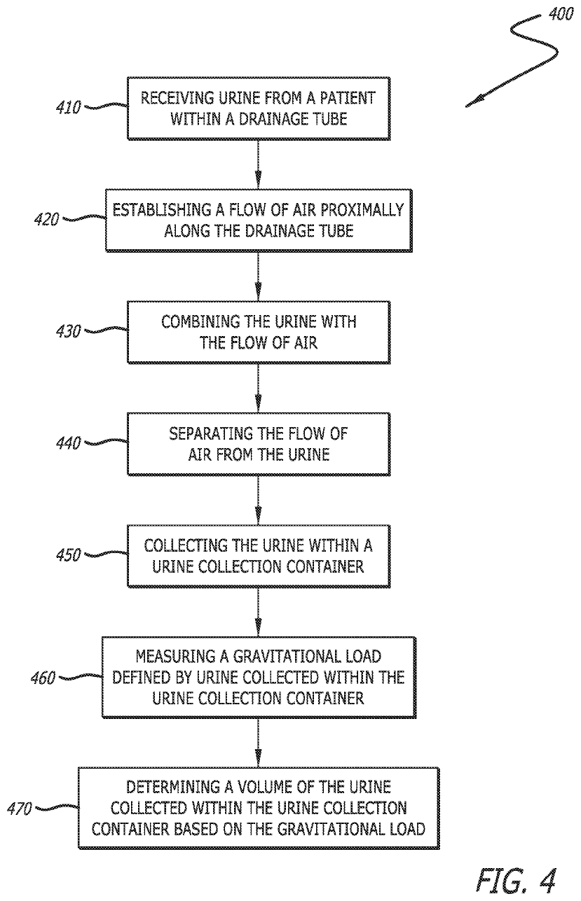

FIG. 4 illustrates a flow chart of an exemplary urinary output monitoring system method. The urinary output monitoring system method (method) 400 may include all or any subset of the following steps, actions, or processes. The method 400 may include receiving urine from a patient within a drainage tube (block 410). The drainage tube may be coupled with and extend proximally away from a urinary catheter.

The method 400 may further include establishing a flow of air proximally along the drainage tube (block 420) where the flow of air takes place along the entire length of the drainage tube between the urinary catheter and an accumulator disposed at a proximal end of the drainage tube. In some embodiments of the method, establishing the flow of air includes activating a vacuum pump fluidly coupled with the accumulator. In some embodiments of the method 400, the flow of air enters the drainage tube by way of the urinary catheter. In some embodiments of the method 400, the urinary catheter is an external female urinary catheter. In some embodiments of the method 400, the flow of air enters the urinary catheter via a non-sealing interface between the urinary catheter and the patient.

The method 400 may further include combining the urine with the flow of air (block 430) such that the flow of air causes the urine to flow proximally along the drainage tube toward the accumulator. The method 400 may further include separating the flow of air from the urine within the accumulator (block 440). The method 400 may further include collecting the urine within a urine collection container (block 450) located downstream of the accumulator. The method 400 may further include measuring a gravitational load defined by urine collected within the urine collection container (block 460). The method 400 may further include determining a volume of the urine collected within the urine collection container based on the gravitational load (block 470).

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A urinary output monitoring system, comprising:
a urine collection assembly, comprising:
a drainage tube defining a distal end and a proximal end;
an accumulator coupled with the drainage tube at the proximal end; and
a urine collection container fluidly coupled with the accumulator;
an automated urinary output monitoring device operatively coupled with the urine collection container, the automated urinary output monitoring device including a console in communication with a sensor of the system, the console including a processor and memory having logic stored thereon that, when executed by the processor, performs operations of the system including determining a volume of urine collected within the urine collection container; and
a vacuum pump fluidly coupled with the accumulator, such that air is removed from the accumulator by the vacuum pump, wherein:
the air removed from the accumulator causes a flow of air combined with a flow of urine along the drainage tube from the distal end to the accumulator, and
the accumulator is configured to separate the flow of air from the flow of urine.

2. The system according to claim 1, wherein the urine collection assembly further comprises a urinary catheter coupled with the drainage tube at the distal end.

3. The system according to claim 2, wherein the flow of air passes through the urinary catheter.

4. The system according to claim 2, wherein the urinary catheter is an external female urinary catheter.

5. The system according to claim 1, wherein:
the accumulator defines a cavity including an upper cavity portion and a lower cavity portion,
the flow of air passes into and out of the upper cavity portion, and
only the flow of urine passes into and out of the lower cavity portion.

6. The system according to claim 1, wherein the accumulator is attached to the automated urinary output monitoring device such that the accumulator is disposed in a vertical orientation.

7. The system according to claim 1, wherein:
the sensor includes a load cell of the automated urinary output monitoring device,
the load cell is operatively coupled with the urine collection container,
the load cell is configured to determine a gravitational load defined by urine collected within the urine collection container, and
determining a volume of urine collected within the urine collection container includes determining the volume of urine based on the gravitational load.

8. The system according to claim 1, wherein the urine collection assembly further comprises a one-way valve disposed in line with a lumen extending between the accumulator and the urine collection container, the one-way valve configured to prevent fluid flow from the urine collection container toward the accumulator.

9. The system according to claim 1, wherein:
the urine collection container is a rigid container, and
the accumulator is rigidly coupled with the urine collection container.

10. The system according to claim 9, wherein the urine collection container includes an air vent configured to a define an atmospheric pressure within the urine collection container.

11. The system according to claim 9, wherein:
the sensor includes a flow meter disposed in line with the lumen extending between the accumulator and the urine collection container,
the flow meter is configured to measure a flow rate of urine passing from the accumulator to urine collection container,
determining a volume of urine collected within the urine collection container includes determining the volume of urine based on the flow rate.

12. The system according to claim 9, wherein the urine collection container includes a detachable lid.

13. A urinary output monitoring system method, comprising:
receiving urine from a patient within a drainage tube coupled with and extending proximally away from a urinary catheter;
establishing a flow of air proximally along the drainage tube between the urinary catheter and an accumulator disposed at a proximal end of the drainage tube;
combining the urine with the flow of air such that the flow of air causes the urine to flow proximally along the drainage tube to the accumulator;
separating the flow of air from the urine within the accumulator; and
collecting the urine within a urine collection container located downstream of the accumulator.

14. The method according to claim 13, wherein establishing the flow of air includes activating a vacuum pump fluidly coupled with the accumulator.

15. The method according to claim 13, further comprising:
measuring a gravitational load defined by urine collected within the urine collection container; and
determining a volume of the urine collected within the urine collection container based on the gravitational load.

16. The method according to claim 13, wherein the flow of air enters the drainage tube by way of the urinary catheter.

17. The method according to claim 13, wherein the urinary catheter is an external female urinary catheter.

18. The method according to claim 17, wherein the flow of air enters the urinary catheter via a non-sealing interface between the urinary catheter and the patient.

19. A urine collection assembly, comprising:
an accumulator defining a closed cavity including an upper cavity portion and a lower cavity portion, the accumulator including an air port in direct fluid communication with the upper cavity portion;
a first drainage tube defining a first distal end and a first proximal end, the first drainage tube coupled with the accumulator at the first proximal end so as to be in direct fluid communication with the upper cavity portion;
a second drainage tube defining a second distal end and a second proximal end, the second drainage tube coupled with the accumulator at the second distal end so as to be in direct fluid communication with the lower cavity portion; and
a urine collection container coupled with the second drainage tube at the second proximal end,
wherein the air port is configured to couple with an air hose of a vacuum pump such that air flow defined by the vacuum pump flows (i) proximally along the first drainage tube and (ii) into and out of the upper cavity portion.

20. The assembly according to claim 19, wherein:
the urine collection container is configured to operatively couple with a load cell of an automated urinary output monitoring device such that urine collected within the urine collection container defines a gravitational load applied to the load cell,
the automated urinary output monitoring device includes a console in communication with the load cell, and
the console includes a processor and memory having logic stored thereon that, when executed by the processor, performs operations of the automated urinary output monitoring device that include determining a volume of the urine collected within the urine collection container based on the gravitational load.

21. The assembly according to claim 19, further comprising:
a female external catheter coupled with the first drainage tube at the first distal end such that, during use, urine excreted by a patient (i) enters the female external catheter, (ii) flows proximally along the first drainage tube and (iii) enters the accumulator.

22. The assembly according to claim 19, wherein the air flow defined by the vacuum pump causes the urine to flow proximally along the first drainage tube.

23. The assembly according to claim 19, wherein, during use, the urine excreted by a patient:
separates from the air flow within the upper cavity portion,
falls from the upper cavity portion to the lower cavity portion,
flows proximally along the second drainage tube, and
enters the urine collection container.
